(19) United States
(12) Patent Application Publication
(10) Pub. No.: US 2025/0244338 A1
(43) Pub. Date: Jul. 31, 2025

(54) METHODS OF ANALYZING SOLUBLE TUMOR NECROSIS FACTOR RECEPTOR 2 (STNFR2) AND USES THEREOF

(71) Applicants: AION HEALTHSPAN, INC., Miami, FL (US); University of Miami, Miami, FL (US)

(72) Inventors: Giacomo Lanzoni, Miami, FL (US); Dimitrios Kouropis, Miami, FL (US); Camillo Ricordi, Miami, FL (US)

(21) Appl. No.: 18/833,530

(22) PCT Filed: Jan. 27, 2023
(86) PCT No.: PCT/US2023/011710

§ 371 Date: Jul. 26, 2024

Related U.S. Application Data
(60) Provisional application No. 63/303,585, filed on Jan. 27, 2022.

(51) Int. Cl. G01N 33/68; A61K 35/28; A61P 37/02; G01N 33/50

(52) U.S. Cl. CPC G01N 33/6863; A61K 35/28; A61P 37/02; G01N 33/5005; G01N 2333/7151

(57) ABSTRACT

The present disclosure relates to methods of testing immunomodulatory activity of cells, including, for example, mesenchymal stem cells and uses of said cells that are determined as having immunomodulatory activity for treating COVID-19 related acute respiratory distress syndrome (ARDS). Disclosed herein are in vitro methods of evaluating mesenchymal stem cells for their effective immunomodulatory effects in vivo.

### CROSS-REFERENCE

This application claims priority to U.S. Provisional Application No. 63/303,585, filed Jan. 27, 2022, which is incorporated by reference in its entirety.

### FIELD

The present disclosure relates to methods of determining immunomodulatory activity of cells and the uses for treating inflammatory diseases.

### BACKGROUND

Safety and efficacy of mesenchymal stem cell (MSC)-based therapies is being investigated in a number of clinical trials for various disorders including inflammatory, immune, autoimmune, musculoskeletal, cardiovascular, neurodegenerative, and gastrointestinal diseases. However, initial results from many such studies reveal that these cell therapies have a substantial degree of variability with cases of non-reproducibility in clinical observation.

What is needed are novel in vitro methods of evaluating MSCs for their effective immunomodulatory effects in vivo.

### SUMMARY

An embodiment of the disclosure is methods for testing immunomodulatory activity of a plurality of cells, comprising
a. separating the plurality of cells into a first group of cells and a second group of cells:
b. culturing the first group of cells in a basal condition and the second group of cells in an inflammatory condition:
c. collecting the culture supernatant of the first group of cells, the culture supernatant of the second group of cells, the cells of the first group of cells, and the cells of the second group of cells:
d. determining the level of soluble TNFR2 protein in the culture supernatant of the first group of cells and the culture supernatant of the second group of cells:
e. normalizing the level of the soluble TNFR2 protein of the first group of cells and the second group of cells with the total protein level of the respective first group of cells and second group of cells collected in step c:
f. calculating an inflammatory stimulation index (ISI) by dividing the normalized level of the soluble TNFR2 protein of the second group of cells by the normalized level of the soluble TNFR2 protein of the first group of cells; and
g. determining that the plurality of cells have responsivity to inflammatory stimulation and immunomodulatory activity if the ISI is higher than 1.

In some embodiments, the plurality of cells comprise human mesenchymal stem cells, or mesenchymal stromal cells, or medicinal signaling cells. In some embodiments, the cells are derived from postnatal adipose tissue, infrapatellar fat pad, postnatal bone marrow, postnatal endometrium, perinatal umbilical cord, perinatal chorion, perinatal amniotic membrane, or perinatal placenta.

In some embodiments, the inflammatory condition comprises the presence of TNFα and/or IFNγ. In some embodiments, the inflammatory condition further comprises the presence of TNFβ, IL-1β, or connective tissue growth factor (CTGF).

An embodiment of the disclosure is a method of treating COVID-19-related acute respiratory distress syndrome (ARDS) in a subject in need thereof, comprising administering to the subject a plurality of cells, wherein the cells are determined as having immunomodulatory activity using the method of any preceding aspect. In some embodiments, the plurality of cells having immunomodulatory activity are determined as having the inflammatory stimulation index (ISI) higher than 1.

An embodiment of the disclosure is a method of treating an inflammatory disorder and/or fibrosis in a subject in need thereof, comprising administering to the subject a plurality of cells, wherein the cells are determined as having immunomodulatory activity using the method of any preceding aspect. In some embodiments, the plurality of cells having immunomodulatory activity are determined as having the inflammatory stimulation index (ISI) higher than 1.

An embodiment of the disclosure is a method of treating an inflammatory condition in a subject in need thereof, comprising administering to the subject a plurality of cells, wherein the cells are determined as having immunomodulatory activity using the method of any preceding aspect. In some embodiments, the plurality of cells having immunomodulatory activity are determined as having the inflammatory stimulation index (ISI) higher than 1.

An embodiment of the disclosure is a method of treating a fibrotic condition characterized in a subject in need thereof, comprising administering to the subject a plurality of cells, wherein the cells are determined as having immunomodulatory activity using the method of any preceding aspect. In some embodiments, the plurality of cells having immunomodulatory activity are determined as having the inflammatory stimulation index (ISI) higher than 1.

An embodiment of the disclosure is a method of treating a condition characterized by an increase in TNF in a subject in need thereof, comprising administering to the subject a plurality of cells, wherein the cells are determined as having immunomodulatory activity using the method of any preceding aspect. In some embodiments, the plurality of cells having immunomodulatory activity are determined as having the inflammatory stimulation index (ISI) higher than 1.

In some embodiments, the method of any preceding aspect further comprises administering to the subject a therapeutically effective amount of an anti-COVID therapeutic agent.

### BRIEF DESCRIPTION OF DRAWINGS

The accompanying figures, which are incorporated in and constitute a part of this specification, illustrate several aspects described below.

FIG. 1 shows the step of standard dilution preparation.

FIG. 2 shows layout of preparing samples and standards in 96 well plate (see Table 5).

FIG. 3 shows schematic of measuring soluble TNFR2 release via normalized quantification and inflammatory stimulation index (ISI).

FIG. 4 shows soluble TNFR2 (sTNFR2) release by UC-MSC over 3 days in basal culture condition versus inflammatory induction.

FIG. 5 shows inflammatory stimulation index (ISI) of sTNFR2 release by UC-MSC, calculated as the ratio of sTNFR2 release in inflammatory condition over basal condition.

FIG. 6 shows observations in patients. Plasma concentrations of soluble tumor necrosis factor receptor 2 (sTNFR2), tumor necrosis factor alpha (TNFα), and tumor necrosis factor beta (TNFβ) in subjects with COVID-19 acute respiratory distress syndrome (ARDS) (n=24). At day 6, UC-MSC recipients had significantly elevated levels of plasma sTNFR2 and significantly decreased levels of TNFα and TNFβ compared to controls. Data are presented as box and whiskers plots indicating the median values and min to max values, and as scatter plots with lines indicating individual values.

FIG. 7 shows sTNFR2 Inflammatory Simulation Index (ISI) of UC-MSC preparations from different donors and at different culture passages. ISI values were obtained via static sTNFR2 release assay. The histograms depict the mean sTNFR2 ISI from triplicate tests; the error bars indicate the Standard Error of the Mean. A: UC-MSC SCI-St Passage 2; B: UC-MSC SCI-St Passage 4; C: UC-MSC SCI-St Passage 5: D: UC-MSC SCI-R01 Passage 4; E: UC-MSC SCI-R01 Passage 5: F: UC-MSC SCI-MCB-1 batch 1 Passage 2; G: UC-MSC SCI-MCB-1 batch 2 Passage 2: H: UC-MSC SCI-MCB-1 Passage 4.

FIG. 8 shows a diagram for counting cells for Trypan Blue Viability Testing.

### DETAILED DESCRIPTION

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

The term “about” as used herein when referring to a measurable value such as an amount, a percentage, and the like, is meant to encompass variations of +20%, +10%, +5%, or +1% from the measurable value.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

A “control” is an alternative subject or sample used in an experiment for comparison purposes. A control can be “positive” or “negative.”

The term “increased” or “increase” as used herein generally means an increase by a statically significant amount: for example, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease, or any decrease between 10-100% as compared to a reference level.

As used herein, the term “level” refers to the amount of a target molecule in a sample, e.g., a sample from a subject. The amount of the molecule can be determined by any method known in the art and will depend in part on the nature of the molecule (i.e., gene, mRNA, cDNA, protein, enzyme, etc.). The art is familiar with quantification methods for nucleotides (e.g., genes, cDNA, mRNA, etc.) as well as proteins, polypeptides, enzymes, etc. It is understood that the amount or level of a molecule in a sample need not be determined in absolute terms, but can be determined in relative terms (e.g., when compared to a control or a sham or an untreated sample).

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

The terms “treat.” “treating.” “treatment,” and grammatical variations thereof as used herein, include partially or completely delaying, alleviating, mitigating or reducing the intensity of one or more attendant symptoms of infection or condition and/or alleviating, mitigating or impeding one or more symptoms of COVID-19-related acute respiratory distress syndrome (ARDS). Treatments according to the invention may be applied preventively, prophylactically, palliatively or remedially. Prophylactic treatments are administered to a subject prior to onset (e.g., before obvious signs of an infection), during early onset (e.g., upon initial signs and symptoms of an infection), after an established development of an infection. Prophylactic administration can occur for several minutes to months prior to the manifestation of an infection.

### Methods

In some aspects, disclosed herein are methods for testing immunomodulatory activity of a plurality of cells, comprising
a. separating the plurality of cells into a first group of cells and a second group of cells:
b. culturing the first group of cells in a basal condition and the second group of cells in an inflammatory condition:
c. collecting the culture supernatant of the first group of cells, the culture supernatant of the second group of cells, the cells of the first group of cells, and the cells of the second group of cells:
d. determining the level of soluble TNFR2 protein in the culture supernatant of the first group and the culture supernatant of the second group:
e. normalizing the level of the soluble TNFR2 protein of the first group and the second group with the total protein level of the respective groups of cells collected in step c:
f. calculating an inflammatory stimulation index (ISI) by dividing the normalized level of the soluble TNFR2 protein of the second group by the normalized level of the soluble TNFR2 protein of the first group; and
g. determining that the plurality of cells have immunomodulatory activity if the ISI is higher than 1.

It should be understood and herein contemplated that soluble Tumor Necrosis Factor Receptor 2 (soluble TNFR2) is also known as sTNFR2, sTNF-RII, TNFRSF1B, CD120b, TBPII, TNF-R-II, TNF-R75, TNFBR, TNFR1B, TNFR2, TNFR80, p75, p75TNFR, tumor necrosis factor receptor superfamily member 1B, TNF receptor superfamily member 1B, which is the product of gene “TNF Receptor Superfamily Member 1B” or TNFRSF1B.

The term “immunomodulatory activity” used herein refers to activity of decreasing inflammatory activity, including, for example, modulation of hyper-inflammatory (e.g., cytokine storm) or hyper-immune response. In some embodiments, the immunomodulatory activity is an anti-inflammatory effect.

In some embodiments, the plurality of cells comprise human mesenchymal stem cells, or mesenchymal stromal cells, medicinal signaling cells, or multipotent stromal cells. In some embodiments, the cells are derived from organs and tissues such as postnatal pancreatic islets or pancreatic tissue, postnatal adipose tissue, infrapatellar fat pad, postnatal bone marrow, postnatal endometrium, postnatal dental pulp, perinatal umbilical cord, perinatal chorion, perinatal amniotic membrane, or perinatal placenta. In some embodiments, the plurality of cells comprise mesenchymal stem cells.

As used herein, a “mesenchymal stem cell” OR “MSC” is a cell capable of differentiating into the mesenchymal cell lineages (i.e., osteoblasts, chondroblasts and adipocytes). Morphological and functional criteria well-known to those of ordinary skill in the art are used to identify these cells. See, Horwitz et al., supra: Dominici et al., supra: Trivedi P & Hematti P, “Derivation and immunological characterization of mesenchymal stromal cells from human embryonic stem cells,” Exp. Hematol. Jan. 5, 2008; Trivedi P & Hematti P, “Simultaneous generation of CD34+ primitive hematopoietic cells and CD56+ mesenchymal stem cells from human embryonic stem cells cocultured with murine OP9 stromal cells,” Exp. Hematol. 35:146-154 (2007); and US Published Patent Application No. 2006/0008902, each of which is incorporated herein by reference as if set forth in its entirety. Mesenchymal stem cells or MSCs can be recognized by their characteristic mononuclear ovoid, stellate shape or spindle shape, with a round to oval nucleus. The oval elongate nuclei typically have prominent nucleoli and a mix of hetero- and euchromatin. These cells have little cytoplasm, but many thin processes that appear to extend from the nucleus. Mesenchymal stem cells or MSCs can typically stain for one, two, three or more of the following markers: CD29, CD44, CD73, CD90, CD105, CD106 (VCAM), CD166 (ALCAM), and alkaline phosphatase, while being negative for hematopoietic lineage cell markers (e.g., CD14, CD34, or CD45) and endothelial lineage cell markers. (e.g., CD31 and VE-cadherin). Mesenchymal stem cells or MSCs may also express STRO-1 and/or CD146 as a marker.

In some embodiments, the inflammatory condition comprises TNFα and/or IFNγ. In some embodiments, the inflammatory condition further comprises TNFβ, IL-1β, connective tissue growth factor (CTGF). Accordingly, in some examples, the second group of cells are cultured in a cell culture medium comprising TNFα, IFNγ, TNFβ, IL-1β, or CTGF, or any combination thereof.

The cells can be cultured for at least 1 minute, at least 1 hour, at least 1 day, at least 2 days, at least 3 days, at least 4 days, at least 5 days, at least 6 days, at least 7 days, at least 8 days, at least 9 days, at least 10 days, at least 15 days, at least 20 days, at least 30 days, or at least 60 days. In some embodiments, the cells are cultured for at least 1 day. In some embodiments, the cells are cultured for at least 2 days. In some embodiments, the cells are cultured for at least 3 days.

Also disclosed herein is a method of treating COVID-19-related acute respiratory distress syndrome (ARDS) in a subject in need thereof, comprising administering to the subject a plurality of cells, wherein the cells are determined as having immunomodulatory activity using the method disclosed herein.

Accordingly, in some aspect, disclosed herein is a method of treating COVID-19-related acute respiratory distress syndrome (ARDS) in a subject in need thereof, comprising administering to the subject a plurality of cells, wherein the cells are determined as having immunomodulatory activity using a method that comprises:
a. separating the plurality of cells into a first group of cells and a second group of cells:
b. culturing the first group of cells in a basal condition and the second group of cells in an inflammatory condition:
c. collecting the culture supernatant of the first group of cells, the culture supernatant of the second group of cells, the cells of the first group of cells, and the cells of the second group of cells:
d. determining the level of soluble TNFR2 protein in the culture supernatant of the first group and the culture supernatant of the second group:
e. normalizing the level of the soluble TNFR2 protein of the first group and the second group with the total protein level of the respective groups of cells collected in step c:
f. calculating an inflammatory stimulation index (ISI) by dividing the normalized level of the soluble TNFR2 protein of the second group by the normalized level of the soluble TNFR2 protein of the first group; and
g. determining that the plurality of cells have immunomodulatory activity if the ISI is higher than 1.

In some embodiments, the plurality of cells are determined as having the ISI higher than 1.

“ARDS” or “acute respiratory distress syndrome” refers to a disease that occurs when fluid builds up in the tiny, elastic air sacs (alveoli) in the lungs. Symptoms of ARDS include, for example, extreme difficulty breathing, shortness of breath, and/or low oxygen levels in the blood that also produce a range of other symptoms, including confusion, dizziness, excessive sweating, low blood pressure, and rapid heart rate. ARDS can be the consequence of inflammatory changes in the lung alveoli. In some embodiments, the administration of the cells that are determined as having immunomodulatory activity using the methods disclosed herein can mitigate one or more of symptoms including, examples, extreme difficulty breathing, shortness of breath, and/or low oxygen levels in the blood. In some embodiments, the administration of the cells decreases inflammation in the subject. In some embodiments, the administration of the cells decreases a level of SARS-CoV-2 virus in the subject. It should be understood and herein contemplated that the terms “increase” and “decrease” used herein can refer to an increase or decrease as compared to prior to the treatment of the subject or as compared with incidence of such symptom in a general or study population.

Also disclosed herein is a method of treating an inflammatory disorder and/or fibrosis in a subject in need thereof, comprising administering to the subject a plurality of cells, wherein the cells are determined as having immunomodulatory activity using the method disclosed herein.

Accordingly, in some aspect, disclosed herein is a method of treating an inflammatory disorder and/or fibrosis in a subject in need thereof, comprising administering to the subject a plurality of cells, wherein the cells are determined as having immunomodulatory activity using a method that comprises:
a. separating the plurality of cells into a first group of cells and a second group of cells:
b. culturing the first group of cells in a basal condition and the second group of cells in an inflammatory condition:
c. collecting the culture supernatant of the first group of cells, the culture supernatant of the second group of cells, the cells of the first group of cells, and the cells of the second group of cells:
d. determining the level of soluble TNFR2 protein in the culture supernatant of the first group and the culture supernatant of the second group:
e. normalizing the level of the soluble TNFR2 protein of the first group and the second group with the total protein level of the respective groups of cells collected in step c:
f. calculating an inflammatory stimulation index (ISI) by dividing the normalized level of the soluble TNFR2 protein of the second group by the normalized level of the soluble TNFR2 protein of the first group; and
g. determining that the plurality of cells have immunomodulatory activity if the ISI is higher than 1.

In some embodiments, the plurality of cells are determined as having the ISI higher than 1.

In some embodiments, the method of any preceding aspect further comprises administering to the subject a therapeutically effective amount of an anti-COVID therapeutic agent.

In an embodiment, the anti-COVID therapeutic agent is one or more of the following baricitinib, ruxolitinib, tofacitinib, imatinib, fluvoxamine, methylprednisolone, lopinavir, ritonavir, darunavir, favipiravir, remdesivir, sofosbuvir/daclatasvir, nirmatrelvir, budesonide, artesunate, type I interferons, telmisartan, nitazoxanide, niclosamide, bromhexine, dornase alfa, dexmedetomidine, fluoxetine, sabizabulin, ribavirin, molnupiravir, danoprevir, bemnifosbuvir, galidesivir, BCX-4430, opaganib, arbidol, chloroquine, dexamethasone, heparin, nitazoxanide, Tocilizumab, Sarilumab, Levilimab, Siltuximab, Clazakizumab, Sirukumab, Olokizumab, Anakinra, Canakinumab, Mavrilimumab, Lenzilumab, Gimsilumab, Otilimab, TJ003234, Emapalumab, Adalimumab, Infiximab, Secukinumab, Ixekizumab, Risankizumab, Lufotrelvir, Ensovibep, Fenretinide, Rintatolimod, Bemcentinib, Plitidepsin, Emetine hydrochloride, Stannous protoporphyrin, Antroquinonol, Apilimod dimesylate, Brequinar, Brilacidin, Sangivamycin, Tempol, RP-7214, PBI-0451, and Masitinib.

In an embodiment, in a static sTNFR2 release assay, cells are cultured in separate culture vessels and exposed to inflammatory or control (basal) medium. The value of sTNFR2 is then measured in the respective media, normalized, and the ratio between sTNFR2 measured in the conditions is calculated.

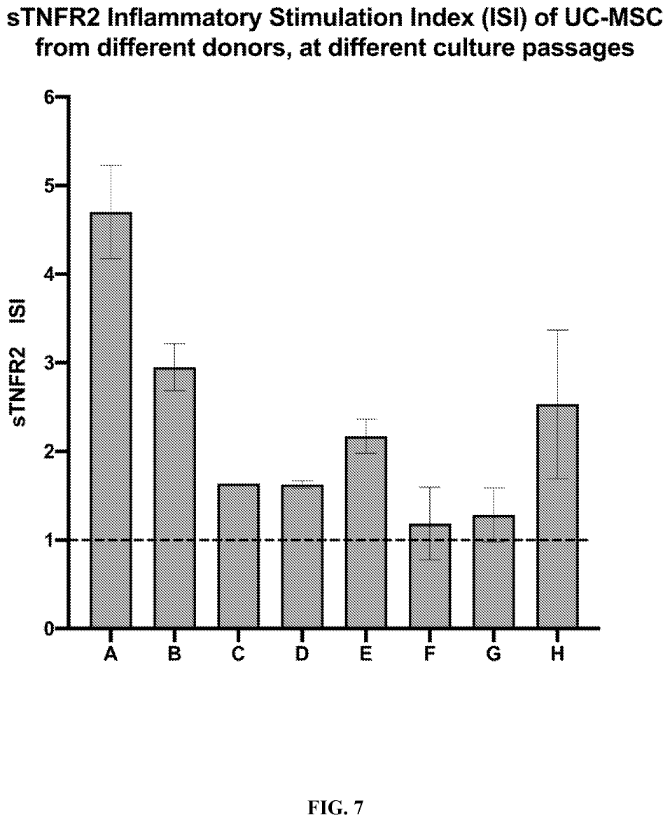

FIG. 7 shows the sTNFR2 Inflammatory Stimulation Index (ISI) with UC-MSC preparations from different donors and at different culture passages. ISI values were obtained via static sTNFR2 release assay. The histograms depict the mean sTNFR2 ISI from triplicate tests: the error bars indicate the Standard Error of the Mean. A: UC-MSC SCI-St Passage 2: B: UC-MSC SCI-St Passage 4: C: UC-MSC SCI-St Passage 5: D: UC-MSC SCI-R01 Passage 4: E: UC-MSC SCI-R01 Passage 5: F: UC-MSC SCI-MCB-1 batch 1 Passage 2: G: UC-MSC SCI-MCB-1 batch 2 Passage 2: H: UC-MSC SCI-MCB-1 Passage 4.

In an embodiment, in a dynamic sTNFR2 release assay, the same cell preparation is maintained immobilized and culture medium is flown around the cells (‘perifusion’). In this embodiment, the same cells can be exposed dynamically to basal and inflammatory condition medium via flow of media with different composition around the immobilized cells, during different time windows. Samples are collected at different timepoints throughout a perifusion assay, the values of sTNFR2 release can be measured in the various samples, and the difference can be calculated in the amount of sTNFR2 measured after exposure to inflammatory medium, compared to basal medium. Instruments are commercially available to perform automated perifusion of media around cells immobilized in a chamber.

The difference in sTNFR2 release, termed ‘Delta sTNFR2’, or ‘ΔsTNFR2’, and the dynamic of the change in sTNFR2 release, are useful when comparing multiple cell preparations.

Normalization of sTNFR2 values can be based on cell protein content at the conclusion of the in vitro experiment, as presented in the preferred embodiment.

In other embodiments, normalization of sTNFR2 can be based on cell number at the initial seeding, or cell seeding density, or cell concentration in culture medium.

In an embodiment, the culture medium contains 10% Platelet Lysate. In other embodiments, the culture medium contains 9%, or 8%, 7%, 6%, 5%, 4%, 3%, 2%, 1% Platelet Lysate. In other embodiments, the culture medium contains no platelet lysate.

In an embodiment, the culture medium is a chemically defined culture medium.

In an embodiment, inflammatory induction is based on the addition of TNFα and IFNγ. In other embodiments, inflammatory induction is based on the addition of TNFα, or TNFβ, or IFNγ, or other inflammatory cytokine.

In certain embodiments, the concentration of TNFα is 0-15000 μg/ml. In certain embodiments, the concentration of TNFα is 0-12500 μg/ml. In certain embodiments, the concentration of TNFα is 0-10000 μg/ml. In certain embodiments, the concentration of TNFα is 0-7000 μg/ml. In certain embodiments, the concentration of TNFα is 0-4000 μg/ml. In an embodiment, the concentration of TNFα is 0-4000 μg/ml. In an embodiment, the concentration of TNFα is 0-3000 μg/ml. In an embodiment, the concentration of TNFα is 0-2500 μg/ml. In an embodiment, the concentration of TNFα is 0-2000 μg/ml. In an embodiment, the concentration of TNFα is 0-1500 μg/ml. In an embodiment, the concentration of TNFα is 0-1000 μg/ml. In an embodiment, the concentration of TNFα is 100-1500 μg/ml. In an embodiment, the concentration of TNFα is 500-2500 μg/ml. In an embodiment, the concentration of TNFα is 500-2000 μg/ml. In an embodiment, the concentration of TNFα is 500-1500 μg/ml. In an embodiment, the concentration of TNFα is 500-1000 μg/ml. In an embodiment, the concentration of TNFα is 500-4000 μg/ml. In an embodiment, the concentration of TNFα is 100-4000 μg/ml.

In certain embodiments, the concentration of TNFβ is 0-20000 μg/ml. In certain embodiments, the concentration of TNFβ is 0)-15000 μg/ml. In certain embodiments, the concentration of TNFβ is 0-10000 μg/ml. In certain embodiments, the concentration of TNFβ is 0-5000 μg/ml. In certain embodiments, the concentration of TNFβ is 500-5000 μg/ml. In certain embodiments, the concentration of TNFβ is 500-10000 μg/ml. In certain embodiments, the concentration of TNFβ is 100-10000 μg/ml. In certain embodiments, the concentration of TNFβ is 100-7500 μg/ml. In certain embodiments, the concentration of TNFβ is 100-5000 μg/ml. In certain embodiments, the concentration of TNFβ is 100-20000 μg/ml. In certain embodiments, the concentration of TNFβ is 500-20000 μg/ml.

In certain embodiments, the concentration of IFNγ is 0-10000 μg/ml. In certain embodiments, the concentration of IFNγ is 0-5000 μg/ml. In certain embodiments, the concentration of IFNγ is 0-2000 μg/ml. In certain embodiments, the concentration of IFNγ is 0-1750 μg/ml. In certain embodiments, the concentration of IFNγ is 0-1500 μg/ml. In certain embodiments, the concentration of IFNγ is 0-1250 pg/ml. In certain embodiments, the concentration of IFNγ is 0-1000 μg/ml. In certain embodiments, the concentration of IFNγ is 500-1500 μg/ml. In certain embodiments, the concentration of IFNγ is 500-1250 pg/ml. In certain embodiments, the concentration of IFNγ is 100-1000 μg/ml. In certain embodiments, the concentration of IFNγ is 100-2000 μg/ml. In certain embodiments, the concentration of IFNγ is 500-2000 μg/ml.

In other embodiments, the cells analyzed for sTNFR2 release are cultured in suspension culture.

In other embodiments, the cells analyzed for sTNFR2 release are cultured in adherence to microcarriers, either without agitation or with agitation, or in suspension.

In certain embodiments, the cells analyzed for sTNFR2 release are adherent to a matrix.

In other embodiments, the cells analyzed for sTNFR2 release are embedded in a material that enables the movement of sTNFR2 through pores in the material.

In certain embodiments, the cells are immobilized by means of adhesion onto a matrix or enclosure into a container, and culture medium is flown around the cells (perifusion) in an in vitro system. The fluid, i.e. culture medium, or solution containing drugs, or a suspension of cells, is flown in an in vitro system to come in contact with the cells. With this method, the dynamic of release of sTNFR2 from the immobilized cells can be analyzed in detail, different samples can be collected over time and/or after exposure to control medium or inflammatory induction medium.

The standardized in vitro method presented here could be utilized to screen cell preparations and select preparations with the greatest potential for clinical efficacy when the goal is to increase the in vivo concentration of sTNFR2, and/or decrease in vivo concentration of TNFα and/or TNFβ.

This method enables selection of a cell population based on in vitro potency related to the release of sTNFR2, with potential to predict in vivo efficacy and effectiveness of the cell population.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Measurement of sTNFR2 Release by Cultured UC-MSC

An embodiment of the disclosure is a method of measuring of sTNFR2 release by cultured UC-MSC, in the presence or absence of inflammatory mediators, via normalized ELISA quantification and Inflammatory Stimulation Index (ISI). This is a representative method and variations in the method are within the scope of the disclosure.

Mesenchymal stem cells (MSC) originate in the human embryo and are considered adult multipotent stem cells. MSC are a heterogeneous subset of stromal stem cells, which can be isolated from the bone marrow, mobilized peripheral blood, cord blood, umbilical cord (UC), placenta, adipose tissue, dental pulp, and even the fetal liver and lungs. UC contains two umbilical arteries (UCA) and one umbilical vein (UCV), both embedded within a specific mucous connective tissue, known as Wharton's jelly (WJ), which is covered by amniotic epithelium. UC is considered medical waste and is collected in a non-invasive manner. Furthermore, the access to UC has not been encumbered with ethical problems. UC-MSC, similarly to MSC derived from other sources, have distinct capacity for self-renewal while maintaining their multipotency, i.e., the ability to differentiate into adipocytes, osteocytes, chondrocytes, neurons, and hepatocytes, although some differentiation abilities are known to be partial. Moreover, MSC have been proposed as a therapeutic modality due to their strong immunomodulatory, anti-inflammatory, and reparative properties.

It was observed that UC-MSC treatment was associated with remarkable clinical benefits, including significant improvements in survival, serious adverse events-free survival, and time to recovery in patients with COVID-19 Acute Respiratory Distress Syndrome (ARDS). It was shown that at Day 6 after infusion, UC-MSC recipients develop significantly increased levels of plasma Soluble TNF Receptor 2 (sTNFR2) and significantly decreased levels of TNFα and TNFβ, compared to controls. Those observations suggested that sTNFR2 plays a mechanistic role in mediating UC-MSC effect on TNFα and TNFβ plasma levels, determining a decrease in inflammation in COVID-19 ARDS.

This study provides the measurement of sTNFR2, a central mediator of the anti-inflammatory effect of UC-MSC treatment, released by cultured UC-MSC, in the presence or absence of inflammatory mediators, via normalized ELISA quantification and Inflammatory Stimulation Index (ISI).

### Definitions

A. GM: Growth Media
B. IIM: Inflammatory Induction Media
C. ISI: Inflammatory Stimulation Index
D. MSC: Mesenchymal Stem Cells
E. Potency: specific ability or capacity of the product to effect a given result.
F. SOP: Standard Operating Procedure
G. sTNFR2: Soluble TNF Receptor 2
H. UC-MSC: Umbilical Cord-Derived Mesenchymal Stem Cells
I. WR: Working Reagent.

### EQUIPMENT AND MATERIALS

A. Equipment
1. Biological Safety Cabinet (BSC)
2. Centrifuge
3. Hemocytometer with cover slip
4. Incubator
5. Micropipettes (2-20 μl, 20-200 μl, 100-1000 μl)
6. Microplate reader (SpectraMax® iD3, Molecular Devices)
7. Microscope
8. Multi-channel Pipette
9. Pipette Aid(s)
10. Refrigerator
11. Timer(s)
B. Supplies:
1. 1.5 ml microcentrifuge tubes
2. 15 ml conical polypropylene tubes
3. 25 cm² Rectangular Canted Neck Cell Culture Flask with Vent Cap
4. 96 well plate
5. Face Masks
6. Gauze
7. Gloves, non-sterile
8. Gloves, sterile
9. Micropipette tips, sterile: 2-200 μl and 100-1000 μl
10. Serological Pipettes, sterile: 5 ml, 10 ml, 25 ml, 50 ml
C. Reagents:
1. CTS™ TrypLE™ Select Enzyme, cGMP-grade, ThermoFisher Scientific
2. DMEM, low glucose, pyruvate, no glutamine, no phenol red, cGMP-grade, ThermoFisher Scientific
3. GlutaMAX 100×, cGMP-grade, ThermoFisher Scientific
4. IFNγ, In vitro use, R&D Systems
5. MEM-NEAA 100×, cGMP-grade, ThermoFisher Scientific
6. Micro BCA Protein Assay Kit, In vitro use, ThermoFisher Scientific
7. PLTGold, cGMP-grade, Mill Creek
8. RIPA Lysis and Extraction Buffer, In vitro use, ThermoFisher Scientific
9. Soluble TNF Receptor 2 Human, ELISA KIT, In vitro use, Abcam
10. TNFα, In vitro use, R&D Systems
11. Human TNF-RII (Soluble) Recombinant Protein, In vitro use, ThermoFisher Scientific
D. Methods:
1. UC-MSC POTENCY ASSAY: Batch Production Record.
2. Media and Reagent Preparation: Inflammatory induction media and Soluble TNF Receptor 2 Human Reagents.
3. Standard Curves Preparation: Soluble TNF Receptor 2 Human Standards and Diluted Albumin (BSA) Standards.

TABLE 1
BSA

| Vial | Volume of Diluent | Volume and Source of BSA | |
|---|---|---|---|
| A | 900 | 100 | Stock |
| B | 800 | 200 | A |
| C | 400 | 400 | B |
| D | 400 | 400 | C |
| E | 400 | 400 | D |
| F | 400 | 400 | E |
| G | 480 | 320 | F |
| H | 400 | 400 | G |
| I | 800 | 0 | Diluent |

TABLE 2
Layout BSA

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | | | | | | |
|---|---|---|---|---|---|---|---|---|
| A | STD A | STD I | — | Batch 42 IIM B | — | — | — | — |

| | | | | | | | |
|---|---|---|---|---|---|---|---|
| B | STD B | Batch 40 IIM A | Batch 42 IIM C | — | — | — | — |
| C | STD C | Batch 40 IIM B | Batch 42 BM A | — | — | — | — |
| D | STD D | Batch 40 IIM C | Batch 42 BM B | — | — | — | — |
| E | STD E | Batch 40 BM A | Batch 42 BM C | — | — | — | — |
| F | STD F | Batch 40 BM B | — | — | — | — | — |
| G | STD G | Batch 40 BM C | — | — | — | — | — |
| H | STD H | Batch 42 IIM A | — | — | — | — | — |

TABLE 3
ELISA

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | | | | | |
|---|---|---|---|---|---|---|---|
| A | STD 1 | Batch 40 IIM A | Batch 42 IIM C | — | — | — | — |
| B | STD 2 | Batch 40 IIM B | Batch 42 BM A | — | — | — | — |
| C | STD 3 | Batch 40 IIM C | Batch 42 BM B | — | — | — | — |
| D | STD 4 | Batch 40 BM A | Batch 42 BM C | — | — | — | — |
| E | STD 5 | Batch 40 BM B | Induced Induction | — | — | — | — |
| | | | Media | | | | |
| F | STD 6 | Batch 40 BM C | Basal Media | — | — | — | — |
| G | STD 7 | Batch 42 IIM A | — | — | — | — | — |
| H | STD 8 | Batch 42 IIM B | — | — | — | — | — |

### ADDITIONAL CONSIDERATIONS

A. In an embodiment, at least two previously trained and qualified cGMP employees should be present throughout this procedure. In an embodiment, all calculations procedures should be verified by the second staff member present.
B. In an embodiment, the manufacturing process described here includes multiple steps and takes 6 days to complete. cGMP Facility personnel involved in product manufacture should log in each step of processing.
C. In an embodiment, cell seeding and expansion should be performed using sterile/aseptic technique. This involves working in a Safety Cabinet (BSC).
D. In an embodiment, the manufacturer, lot number and expiration date of all supplies, media and reagents used in this procedure should be documented.
E. In an embodiment, bring all samples and reagents from sTNFR2 Human ELISA KIT to room temperature (18-25° C.) before use.
F. In an embodiment, opened Microplate Wells or reagents may be store for up to 1 month at −20° C.

### PROCEDURE

### A. Media, Reagents, and Standard Preparation

1. Media preparation
1.1 Growth Media (GM)

To prepare a bottle of media, add the following reagents in the sequence indicated below:
a. 1000 ml DMEM
b. 54 ml PLTGold™ (5% PLTGold)
c. 10.8 ml GlutaMAX Supplement 200 mM
d. 10.8 ml MEM-NEAA, 10 mM Label the media with:
   Name of the media
   Date of preparation
   Date of expiration (14 days following preparation)·
   Initials of the cGMP Facility personnel preparing the media
1.2 Inflammatory Induction Media (IIM)
NOTES: Prepare media prior to use on day 3.
To prepare a 45 ml of IIM, add the following reagents in the sequence indicated.
a. 44.5 ml GM
b. 225 μl IFNγ (Final concentration: 10 ng/ml)
c. 337.5 μl TNFα (Final concentration: 15 ng/ml) Label the media with:
   Name of the media.
   Date of preparation.
   For fresh use
   Initials of the cGMP Facility personnel preparing the media
2. Reagent preparation (Soluble TNF Receptor 2 Human ELISA KIT)

TABLE 4

| Reagent | Preparation |
|---|---|
| 1X Assay Diluent B | 5X Assay Diluent B should be diluted 5-fold with deionized |
| | water before use. |
| 1X Wash Solution | If the 20X Wash Concentrate contains visible crystals, |
| | equilibrate to room |
| | temperature, and mix gently until dissolved. Dilute 20 ml Wash |
| | Buffer into deionized water to yield 400 ml of 1X Wash Buffer. |
| 1X Biotinylated | Briefly spin the Biotinylated anti-human Soluble TNF Receptor |
| Soluble TNF Receptor | 2 vial before use. Add 100 μl of 1X Assay Diluent B into the |
| 2 DetectionAntibody | vial to prepare a detection antibody concentrate. Pipette up and |
| | down to mix gently. The detection antibody concentrate should |
| | be diluted 80-fold with 1X Assay Diluent B prior |
| | to use in the Assay procedure. |
| 1X HRP - | Briefly spin the 500X HRP-Streptavidin concentrate vial and |
| Streptavidin Solution | pipette up and down to mix gently before use. Dilute HRP- |
| | Streptavidin concentrate with 500-fold with 1X Assay Diluent |
| | B. (For example: Add 20 μl of 500X HRP- Streptavidin |
| | concentrate into a tube with 10 ml 1X Assay Diluent B to |
| | prepare a final 500-fold diluted 1X HRP-Streptavidin solution). |
| | The diluted solution should not be stored for next day use. |

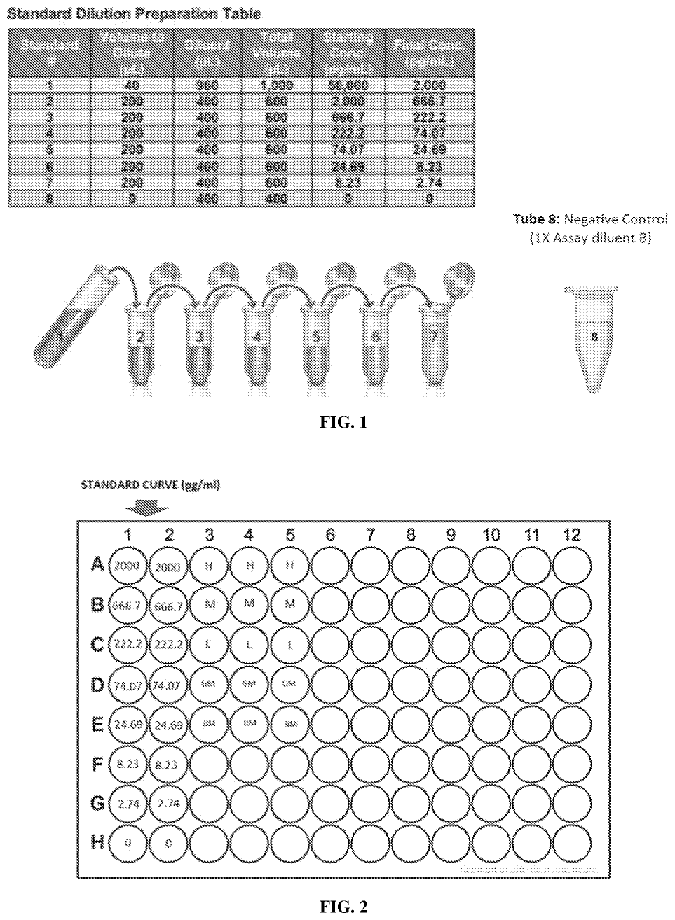

3. Preparation of Soluble TNF Receptor 2 Human Standards
NOTE: Prepare serially diluted standards immediately prior to use according to manufacturer's recommendations. Always prepare a fresh set of standards for every use.
3.1 Reconstitute only 1 vial (1 vial/standard curve).
3.2 Briefly spin the vial of sTNFR2 Standard. Prepare the 50 ng/ml Stock Standard by adding 400 μl of 1× Assay Diluent B into the vial. Mix thoroughly and gently.
3.3 Label tubes #1-7 for serial standard dilution by 1 to 3 dilution factor.
3.4 Prepare Standard #1 by adding 40 μl of the 50 ng/ml Stock Standard, to 960 μl of 1× Assay Diluent B into tube #1. Mix thoroughly and gently.
3.5 Pipette 400 μl of 1× Assay Diluent B into remaining tubes.
3.6 Prepare Standard #2 by adding 200 μl Standard #1 to tube #2 and mix thoroughly.
3.7 Prepare Standard #3 by adding 200 μl Standard #2 to tube #3 and mix thoroughly.
3.8 Using FIG. 2 as a guide, prepare further serial dilutions (see Table 5).
3.9 1× Assay diluent B (Tube 8: Negative control) serves as the zero standard (0 pg/ml) (FIG. 1).

TABLE 5
Preparation of Soluble TNF Receptor 2 Human Standards

| | | | Add 200 | | |
|---|---|---|---|---|---|
| | Stock | 1x Assay | μl from | Total | |
| Standard | Standard | Diluent | Previous | Volume | Final |
| # | (μl) | B (μl) | Standard | (μl) | Concentration |
| 1 | 40 | 960 | 0 | 1000 | 2000 |
| 2 | 0 | 400 | +200 μl | 400 | 666.7 |
| | | | Std #1 | | |
| 3 | 0 | 400 | +200 μl | 400 | 222.2 |
| | | | Std #2 | | |
| 4 | 0 | 400 | +200 μl | 400 | 74.07 |
| | | | Std #3 | | |
| 5 | 0 | 400 | +200 μl | 400 | 24.69 |
| | | | Std #4 | | |
| 6 | 0 | 400 | +200 μl | 400 | 8.23 |
| | | | Std #5 | | |
| 7 | 0 | 400 | +200 μl | 600 | 2.74 |
| | | | Std #6 | | |
| 8 | 0 | 400 | 0 | 400 | 0 |
| (Negative | | | | | |
| Control) | | | | | |

4. Preparation of Diluted Albumin (BSA) Standards
Each 1 mL ampule of 2.0 mg/mL Albumin Standard is sufficient to prepare a set of diluted standards such that three replicates of each dilution can be included in the Test Tube Procedure. A fresh set of standards should be prepared for every use.
4.1 Prepare a set of protein standards using distilled water. Use Table 6 as a guide.

TABLE 6
Preparation of Diluted Albumin (BSA) Standards

| | Volume | | Final BSA | |
|---|---|---|---|---|
| Vial | of Diluent | Volume and Source of BSA | Concentration | |
| A | 4.5 mL | 0.5 mL of Stock | 200 | μg/mL |
| B | 8.0 mL | 2.0 mL of vial A dilution | 40 | μg/mL |
| C | 4.0 mL | 4.0 mL of vial B dilution | 20 | μg/mL |
| D | 4.0 mL | 4.0 mL of vial C dilution | 10 | μg/mL |
| E | 4.0 mL | 4.0 mL of vial D dilution | 5 | μg/mL |
| F | 4.0 mL | 4.0 mL of vial E dilution | 2.5 | μg/mL |
| G | 4.8 mL | 3.2 mL of vial F dilution | 1 | μg/mL |
| H | 4.0 mL | 4.0 mL of vial G dilution | 0.5 | μg/mL |

| | | | |
|---|---|---|---|
| I | 8.0 mL | 0 | 0 μg/mL = Blank |

4.2 Use the following formula to determine the total volume of Working Reagent (WR) required:

$$[ ( \# standard \times 2 replicates ) + ( \# unknowns \times 3 replicates ) ] \times ( 150 μl of WR ) = total volume WR required .$$

4.3 Prepare the volume WR required by mixing 25 parts of Micro BCA Reagent A (MA) and 24 parts of Reagent B (MB) with 1 part of Micro BCA Reagent C (MC) (25:24:1).

NOTE: WR is stable for one day when stored in closed container at room temperature.

### B. Cell Seeding and Expansion

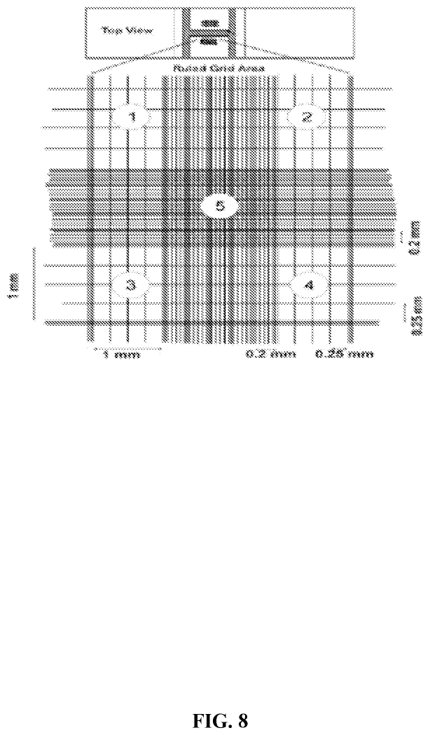

1. Thawing UC-MSC vial or segment(s) and Cell expansion
1.1 Remove 1 vial or 2-3 segments containing UC-MSC from cryo-storage.
1.2 Place the sample in a specimen bag and quickly thaw it by gently swirling it in the 37° C. water bath (until small crystals of ice remains in the vial or segment(s)). Handle the cells gently to avoid mechanical injury. This should take no more than 3 minutes.
1.3 Once the sample is thawed, wipe it down using a sterile Alcohol prep pad.
1.4 Bring the sample inside a BSC and slowly, transfer the cells with a syringe into a sterile 50 ml conical tube.
1.5 Slowly, in a drop-wise fashion, add 10 ml of room temperature GM to the same 50 mL conical tube.
1.6 Centrifuge cells at 500×g for 10 minutes, at room temperature, with the brake set to “high”.
1.7 Remove cells from the centrifuge and gently aspirate off the supernatant.
1.8 Re-suspend the pellet in 10 ml of GM, equilibrated to room temperature.
1.9 Perform a cell count and viability (“Viability Testing Using Trypan Blue or Propidium Iodide (PI) and Thiazole Orange (TO)”. Record the results. The trypan blue viability test is based on the ability of the cell membrane of live (viable) cells to exclude (trypan blue), and nonviable cells to take up the dye. Cells are evaluated under the microscope and a count of both viable and non-viable cells is performed to determine the percent viability. Trypan Blue should be filtered before use. Cells should be counted as soon as the dye is added. Viable cells will begin to take up the dye within 2-3 minutes, which will result in lower viability.
a. Reagents
   0.4% Trypan Blue, filtered, Sigma or equivalent
   Phosphate Buffered Saline, (PBS), MediaTech or equivalent
   70% Ethanol
b. Procedure
Viability on Fresh Cells
1. The following procedure is for fresh cells prepared in a suspension.
2. Ensure that the hemacytometer and cover glass are cleaned thoroughly and air-dried. Use 70% alcohol and a lint-free wipe.
3. Place coverslip on hemacytometer so that it partially covers both V slashes.
4. Using a micropipette, thoroughly mix the fresh cells and place 100 μl in a small tube.
5. Add 100 μl 0.4% trypan blue. If the cell product is too concentrated dilute with PBS prior to the addition of trypan blue and mix well.
6. Charge hemacytometer as follows:
a. Use a pipette to place a drop of the cells/trypan blue mixture between the cover glass and counting chamber on one of the V slashes without disturbing the coverslip. Slowly fill the area completely and remove the pipette before the solution can overflow at the edges of the chamber section.
b. Do not overfill or underfill. If the chamber is overloaded or air bubbles are visible, clean the chamber and start again, rather than attempting to remove excess liquid.
7. Rotate to the 4× objective and carefully place the hemacytometer on the microscope stage.
8. Count cells after approximately a 1 minute incubation and prior to 3 minutes. After 2-3 minutes of incubation the viable cells may start to take up the dye, producing an inaccurate count.
9. Viable cells will remain unstained, while dead cells will appear blue after taking up the dye.
10. Count the number of stained nucleated cells versus unstained nucleated cells in a total of 100 random cells. These counts can be performed on any of the quadrants numbered 1-5 in FIG. 8.
11. Record data.
12. Calculate the percent viability using the following formula:

$$\% Viable cells = Number of viable cells \times 100$$

$$Total \# of cells counted$$

### Viability of Thawed Cells

1. Rapidly thaw cryopreserved cells.
2. Follow steps #2 to #12 from Viability on Fresh Cells procedure.
1.10 Seed the cells in six 25 cm² flasks (3 flask per condition: “Basal” and Inflammatory induction”) at a concentration of 500,000 viable cells per flask (20.000 cells/cm²), resuspended in 5 mL GM per flask.
1.11 The cells are cultured in GM for 72 h (3 days) in a 37° C., 5% CO₂ incubator.

### C. Inflammatory Induction

1. Inflammatory induction (TNFα/IFNγ)
1.1 Prepare IIM prior to use and warm up at 37° C.
1.2 Remove all the media from the flasks.
1.3 Add 5 ml of pre-warmed GM to one flask, mark the flask as ‘Basal’.
1.4 Add 5 ml of pre-warmed IIM to the other flask, mark the flask as ‘Inflammatory induction’.
1.5 Place tissue culture flasks back into the 37° C. incubator with 5% CO₂ for 3 days.

### D. Potency Assay

After day 3 of induction:
1. sTNFR2 MEASUREMENT
NOTE: It is recommended to assay all standards in duplicate, and samples in triplicate. The 96 well plate strips included in the kit are supplied ready to use.
1.1 Collect culture supernatants in a 15 ml tubes.
1.2 Centrifuge the supernatant at 1,500 rpm for 5 minutes to remove debris.
1.3 Gently pour supernatant into new 15 ml tubes.
1.4 Dilute the supernatant 5-fold with the 1× Assay Diluent B (Reagent Preparation, section V, A, 2) from the sTNFR2 Human ELISA KIT (Abcam).
1.5 Add 100 μL of each standard in duplicates (see Standard Preparation section V, A, 3) and each sample (UC-MSC samples, TNFR2 Standards, Media) in triplicates into appropriate wells (FIG. 2). Cover well and incubate for overnight at 4° C. with gentle shaking.
1.5.1 TNFR2 Standard:
a) H defined as a high concentration (H)
b) M defined as a mid-concentration (M)
c) L defined as a low concentration (L)
1.5.2 Blank media:
a) GM
b) IIM
1.5.3 Supernatant (Test samples):
a) Supernatant GM (sGM)
b) Supernatant IIM (sIIM)
1.6 The next day, discard the solution and wash 4 times with 1× Wash Solution. Wash by filling each well with 1× Wash Solution (300 μL) using a multi-channel Pipette or auto washer. After the last wash, remove any remaining Wash Buffer by decanting. Invert the plate and blot it against clean paper towels.
1.7 Add 100 μL of 1× Biotinylated Soluble TNF Receptor 2 Detection Antibody (Reagent Preparation, section V, A, 2) to each well. Incubate for 1 hour at room temperature with gentle shaking.
1.8 Discard the solution. Repeat the wash as in step 1.6.
1.9 Add 100 μL of 1×HRP-Streptavidin solution to each well. Incubate for 45 minutes at room temperature with gentle shaking.
1.10 Discard the solution. Repeat the wash as in step 1.6.
1.11 Add 100 μL of TMB One-Step Substrate Reagent to each well. Incubate for 30 minutes at room temperature in the dark with gentle shaking.
1.12 Add 50 μL of Stop Solution to each well.
1.13 Load the plate in the SpectraMax® iD3 and read the plate at 450 nm immediately.
2. PROTEIN QUANTITATION

NOTE: sTNFR2 secretion is normalized to cell lysate total protein content. For this, the cell lysate total protein content is quantified with the ‘Micro BCA Protein Assay Kit’ (ThermoFisher Scientific).
2.1 Protein lysate
a. Briefly, after supernatant removal, wash the cell monolayers twice with 5 ml cold DPBS.
b. Add 1 ml of cold RIPA buffer (4° C.) with a micropipette to each 25 cm² flasks.
c. Incubate the flasks at 4° C. for 5 minutes.
d. Collect cell lysates and centrifuged at 10,000 rpm for 5 mins to remove cell debris.
e Upon centrifugation, transfer cell lysates to new microcentrifuge tubes and further processed for protein quantitation.
2.2 Protein quantitation with Micro BCA Protein Assay
a. Prepare a bovine serum albumin standard curve (0-200 μg/ml) as described herein.
b. Dilute Cell lysates 1:100 with distilled water.
c. Add 150 μL of each standard in duplicates or diluted cell lysate in triplicates into 96 well plate.
d. Add into each well 150 μL of the WR (25:24:1, Reagent MA: MB: MC).
e. Mix thoroughly on a plate shaker for 30 seconds.
f. Incubate the plate at 37° C. for 2 hours.
g. Measure the absorbance at 562 nm on a microplate reader.

### E. Analysis

1. The assay is performed on three sets of induced and non-induced flasks, and results analyzed using the SoftMax Pro Software.
2. sTNFR2 ELISA:
2.1 sTNFR2 concentrations for each sample are calculated using the assay standard curve (0-2,000 μg/ml) fit to a 4 parametric logistic regression.
2.2 Triplicate well values of the two blank media samples and six test samples (3x induced and 3x non-induced) are averaged to obtain average sTNFR2 concentrations for each blank/flask.
2.3 There should be a total of eight averages, one for each flask, and one for each blank media.
3. Total Protein BCA Assay:
3.1 Cell lysate total protein concentrations for each sample are calculated using the Micro BCA Protein Assay standard curve fit to a linear regression.
3.2 Triplicate well values of the six test samples (3x induced and 3x non-induced) are averaged to obtain average Total Protein for each flask.
3.3 There should be a total of six average one for each flask.
4. Normalized sTNFR2 Calculation:
4.1 For all flasks (both the UC-MSC basal and UC-MSC inflammatory induction groups), the sTNFR2 concentrations are normalized by dividing each flask sTNFR2 value by the corresponding Total Protein concentration value.
4.2 The Normalized sTNFR2 in basal culture condition is then calculated by averaging the UC-MSC basal normalized sTNFR2 concentrations. The resulting value should be ≥0.2 (pg/mL)/(ug of total protein).
5. Inflammatory Stimulation Index (ISI) Calculation:
5.1 The ISI is calculated for each set of flasks, as the ratio of induced normalized sTNFR2 concentration to non-induced normalized sTNFR2 concentration.
5.2 The three ISI are averaged, and the resulting value should be ≥1.5.

### Notes

A. The total cell protein content is calculated based on the volume of the cell lysate used (RIPA, 1.5 ml) and on the base of the dilution factor (1:100).

### Quality Control

A. To evaluate the potential for false positive results on the quantitation of the sTNFR2, analyze media samples (Growth Media and Inflammatory Induction Media).
B. An external sTNFR2 standard with concentrations ranging across the standard curve is also run to ensure the ELISA kit is working appropriately. These values should be within 20% of the theoretical concentration.
C. The % CV of the induced and non-induced ELISA replicates should be ≤20% to ensure an accurate result. If the % CV is >20%, one value can be dropped as an outlier.
D. The standard curve should be fit to a 4-parameter logistic regression and have an R2 value of ≥0.98 to be considered acceptable. If the R2 value is <0.98, values can be dropped as outliers as long as there is at least one value for each concentration of standard.
E. All equipment used should be maintained.

### Example 2. In Vitro Potency Assay for Immunomodulatory Cells Based on the Analysis of Soluble Tumor Necrosis Factor Receptor 2 (sTNFR2) Release

In vitro potency assay for immunomodulatory cells based on the analysis of soluble Tumor Necrosis Factor Receptor 2 (sTNFR2) release. This is a representative assay and variations in the assay protocol are within the scope of the disclosure.

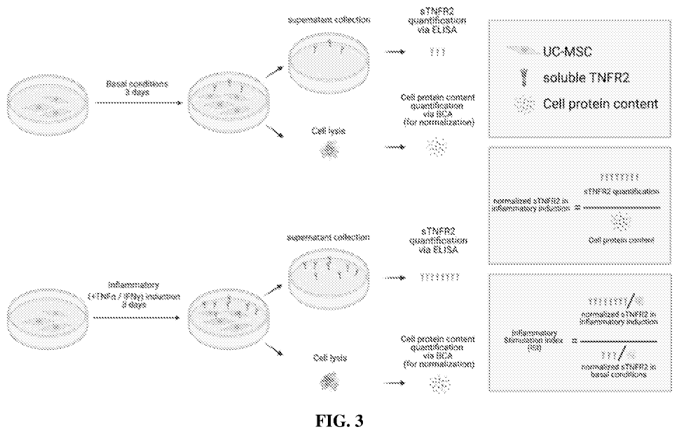

The study herein has developed and qualified a biologically relevant in vitro assay to determine the potency of immunomodulatory cells, such as Mesenchymal Stem Cells. The potency assay is focused on the measurement of soluble Tumor Necrosis Factor Receptor 2 (sTNFR2) released by the cells. It is based on sTNFR2 quantification via ELISA, normalized with cell protein content, and calculation of the Inflammatory Stimulation Index (ISI) of sTNFR2 released by the cells. The ISI is calculated as the ratio of sTNFR2 release in inflammatory induction over basal condition. The assay is performed with in vitro cultured cells. In an embodiment, the cells to be analyzed with the assay are Umbilical Cord-derived Mesenchymal Stem Cells (UC-MSC). The basal condition corresponds to culturing the cells in the same medium utilized for the generation of the final cell product. The inflammatory induction derives from addition of TNFα (15 ng/ml) and IFNγ (10 ng/ml) in the medium of these cultures. The cells are maintained in basal conditions or under inflammatory induction for a specific amount of time of culture, and in a preferred embodiment for 3 days of culture. The supernatant is then collected and tested with a commercially available kit for sTNFR2 quantification (e.g., Abcam Soluble TNFR2 Human ELISA KIT, Abcam, Cat #ab100643). For normalization based on total cell protein content, the cells are lysed with RIPA Lysis and Extraction buffer (e.g. ThermoFisher Scientific, Cat #89900) and protein content is obtained with the BCA method (e.g., with the Micro BCA Protein Assay Kit, ThermoFisher Scientific, Cat #23235). The assay is described in FIG. 3. The SOP for the assay is provided herein above in Example 1.

### Data of the Potency Assay

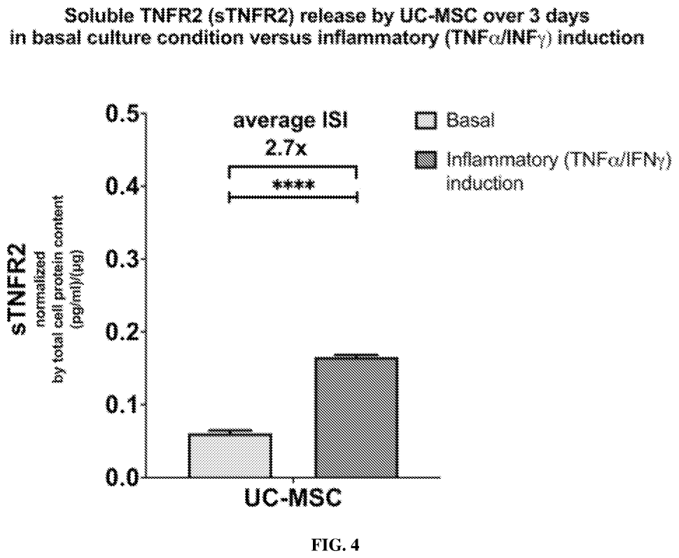

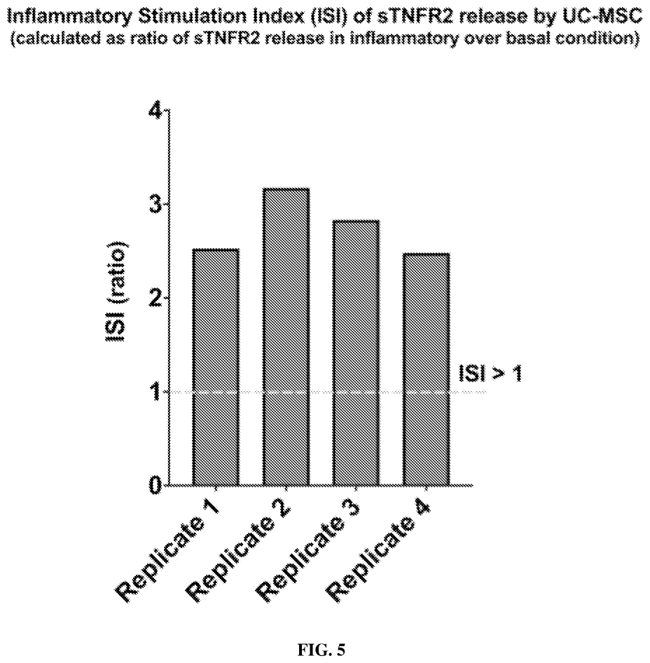

The methods described in Example I have been applied, with UC-MSC thawed from the stage of “UC-MSC Final Product (Batch, Cryopreserved)”, manufactured at the Diabetes Research Institute cGMP facility. The experiment quantified the sTNFR2 release by UC-MSC over 3 days in basal culture condition versus inflammatory (TNFα/IFNγ) induction. Results are presented in Table 7, FIG. 4, and FIG. 5.

TABLE 7

| | | Cell | | |
|---|---|---|---|---|
| | sTNFR2 | lysate | Normalized | |
| UC-MSC | replicates | protein | sTNFR2 | Inflammatory |
| Culture | concentrations | content | (pg/ml)/μg | Stimulation |
| Conditions | (pg/ml) | (μg) | protein) | Index (ISI) |
| Basal | 52.7 | 788 | 0.067 | 1 |
| Condition | 41.7 | | 0.053 | 1 |
| | 43.4 | | 0.055 | 1 |
| | 53.4 | | 0.068 | 1 |
| Inflammatory | 97.5 | 576 | 0.169 | 2.527 |
| (TNFα/IFNγ) | 96.7 | | 0.168 | 3.174 |
| Induction | 90.0 | | 0.156 | 2.832 |
| | 96.9 | | 0.168 | 2.479 |

### Biological Relevance of the Assay in Relation to Patients

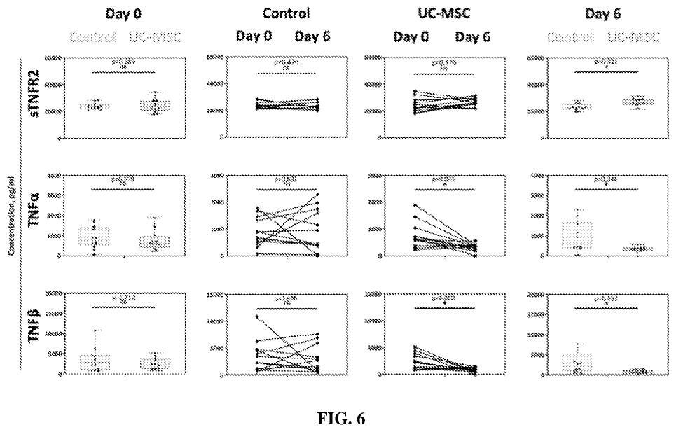

The hyperinflammatory response in COVID-19 patients with Acute Distress Respiratory Syndrome (ARDS) is characterized by high serum levels of pro-inflammatory mediators, including tumor necrosis factor (TNF) α and β. These two molecules, implicated in ARDS pathophysiology, bind to TNFR2. A soluble form of TNFR2 was reported to have inhibitory effect on TNF functions. The study herein investigated the plasma levels of TNFα, TNFβ, and soluble TNFR2 (sTNFR2) in both UC-MSC treatment and control groups in the Phase 1/2a clinical trial for COVID-19 ARDS. sTNFR2 was increased in patients of the UC-MSC treatment group, compared to patients in the control group, at day 6 (see FIG. 6). TNFα and TNFβ were found to be decreased at day 6. The observations are presented in FIG. 6.

### Methods (Observations in Patients)

Blood samples were obtained from clinical trial randomized subjects at day 0 (before infusion) and day 6 (3 days after second infusion). Briefly, whole blood was collected into EDTA treated tubes, transferred on ice, and processed for plasma separation within 2 hours. Whole blood was centrifuged at 2,000 g for 15 min at 4° C., and plasma was collected and stored at −80° C. until processing. A quantitative multiplex protein array (Ray Bio® Q-Series, Ray Biotech) was utilized to determine the TNFR2, TNFα, TNFβ plasma levels (pg/ml) in all samples at the same time, following manufacturer's instructions. The fluorescent signals were visualized via a Cy3 wavelength laser scanner and converted to concentrations using the standard curve generated per array.

Statistical analysis was performed using two sample T-tests and nonparametric Wilcoxon two-sample tests. Signed rank tests were used for paired comparisons examining changes between timepoints within group. All tests were two-sided, with statistical significance established with p<0.05. Data are presented with means and standard errors of the mean.

### Results (Observations in Patients)

Patients in UC-MSC and control groups showed no significant differences in baseline protein levels. In control group, sTNFR2, TNFα and TNFβ levels were not significantly different between days 0) and 6. TNFα and TNFβ levels decreased significantly between day 0 and day 6, (p=0.005 and p=0.002, respectively). Comparisons between groups on day 6 demonstrated significantly lower levels in UC-MSC group compared to control group of TNFα (319±40 vs 950±226 μg/ml, p=0.048) and TNFβ (810±126 vs 2,944±735 μg/ml, p=0.032). sTNFR2 showed significantly higher levels in the UC-MSC group compared to control on day 6 (26,609±846 μg/ml vs 23,111±760 μg/ml, p=0.021). See FIG. 6.

In the recently completed phase 1/2a clinical trial, UC-MSC treatment was associated with accelerated clinical recovery in patients with COVID-19 ARDS. Provided herein is molecular evidence of differences in a key underlying immune/inflammatory mediator axis that help explain those results. At day 6, UC-MSC recipients had significantly elevated levels of plasma sTNFR2 and significantly decreased levels of TNFα and TNFβ, compared to controls. TNF receptor-based drugs have been tested to treat chronic inflammatory diseases, and similarly can be beneficial for the hyperinflammation attenuation in severe COVID-19 patients. TNF blockade is clinically effective as it results in rapid reduction of circulating interleukin (IL)-1 and IL-6 levels (<12 hours), and reduction in adhesion molecules and vascular endothelial growth factor (VEGF) that strongly affect leukocytes trafficking and capillary permeability in inflamed tissues. Studies showed that upon anti-TNF therapy, TNF concentration in inflamed tissues is reduced as it passes into blood circulation bound to the anti-TNF antibodies.

Furthermore, sTNFR2 is capable of binding TNF and neutralize TNF-induced cytotoxicity and immune-reactivity, modulating inflammatory reactions. For instance, higher sTNFR2 levels lead to decreased T cell activation and gradual production of regulatory T cells (Tregs). On this basis, studies showed that expression of TNFR2 by MSC is correlated to their higher Foxp3+T reg induction capacity. Therefore, the findings herein show a key mechanism of UC-MSC effect, whereas sTNFR2 blood plasma levels can be a predictor for COVID-19 ARDS progression and clinical outcome after therapy.

Based on the observations in patients, described here above, and based on the observations of the potency assay, described in previous paragraphs, the potency assay developed herein is biologically relevant.

### Assurance of Potency

This potency assay, measurement of soluble TNFR2 release via normalized quantification and Inflammatory Stimulation Index (ISI), is utilized for assurance of potency of the product to be used in a proposed Phase 2b/3 study. The criteria for assurance of potency of each batch of UC-MSC utilized for the Phase 2b/3 study are:
soluble TNFR2 (sTNFR2) release, normalized by total cell protein content, over 3 days culture >0.01 (pg/mL)/(ug)·
Inflammatory Stimulation Index (ISI)>1
In an embodiment, soluble TNFR2 (sTNFR2) release, normalized by total cell protein content, over 3 days culture in basal condition >0.02 (pg/mL)/(ug)·
Inflammatory Stimulation Index (ISI)>1.5
In an embodiment, soluble TNFR2 (sTNFR2) release, normalized by total cell protein content, over 3 days culture under Inflammatory Induction >0.03 (pg/mL)/(ug)·
Inflammatory Stimulation Index (ISI)>1.5

This disclosure addresses the need for a potency assay for immunomodulatory cells (or their secretome) that modify the Tumor Necrosis Factor (TNF) inflammatory pathway in humans. Types of immunomodulatory cells include Mesenchymal Stem Cells, Mesenchymal Stromal Cells, Medicinal Signaling Cells (MSC).

This in vitro potency assay is based on the measurement of the release of soluble Tumor Necrosis Factor Receptor 2 (sTNFR2) in vitro in basal or inflamed conditions, to predict in vivo immunomodulatory function. Currently, safety and efficacy of MSC-based therapies is being investigated in a number of clinical trials for various disorders including inflammatory, immune, autoimmune, musculoskeletal, cardiovascular, neurodegenerative, and gastrointestinal diseases. However, initial results from many of such studies reveal that these cell therapies have a substantial degree of variability with cases of non-reproducibility in clinical observation. Most importantly, the inconsistent evidence potentially relates to intrinsic differences in the cell-based products used, including lack of standardized features in the preparations reflected in potency discrepancies. Therefore, herein shown is a rapid and accurate method to a priori qualitative evaluate MSC batches in vitro for effective immunomodulatory actions in vivo.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A method of testing immunomodulatory activity of a plurality of cells, comprising:
a. separating the plurality of cells into a first group of cells and a second group of cells:
b. culturing the first group of cells in a basal condition and the second group of cells in an inflammatory condition;
c. collecting the culture supernatant of the first group of cells, the culture supernatant of the second group of cells, the cells of the first group of cells, and the cells of the second group of cells;
d. determining the level of soluble Tumor Necrosis Factor Receptor 2 (TNFR2) protein in the culture supernatant of the first group of cells and in the culture supernatant of the second group of cells;
e. normalizing the level of the soluble TNFR2 protein of the first group of cells and the second group of cells with the total protein level of the respective first group of cells and second group of cells collected in step c;
f. calculating an inflammatory stimulation index (ISI) by dividing the normalized level of the soluble TNFR2 protein of the second group of cells by the normalized level of the soluble TNFR2 protein of the first group of cells; and
g. determining that the plurality of cells have immunomodulatory activity if the ISI is higher than 1.

2. The method of claim 1, wherein the plurality of cells comprises human mesenchymal stem cells, mesenchymal stromal cells, or medicinal signaling cells.

3. The method of claim 1 or 2, wherein the plurality of cells are derived from postnatal adipose tissue, infrapatellar fat pad, postnatal bone marrow, postnatal endometrium, perinatal umbilical cord, perinatal chorion, perinatal amniotic membrane, or perinatal placenta.

4. The method of any one of claims 1-3, wherein the inflammatory condition comprises the presence of Tumor Necrosis Factor α (TNFα) or Interferon γ (IFNγ).

5. The method of claim 4, wherein the inflammatory condition further comprises the presence of TNFβ, IL-1β, or connective tissue growth factor (CTGF).

6. A method of treating COVID-19-related acute respiratory distress syndrome (ARDS) in a subject in need thereof, comprising administering to the subject a plurality of cells, wherein the plurality of cells are determined as having immunomodulatory activity using the method of any one of claims 1-5.

7. The method of claim 6, wherein the plurality of cells are determined as having the inflammatory stimulation index (ISI) higher than 1.

8. The method of claim 6 or 7, further comprising administering to the subject a therapeutically effective amount of an anti-COVID therapeutic agent.

9. A method of treating a condition of at least one of an inflammatory disorder or fibrosis in a subject in need thereof, comprising administering to the subject a plurality of cells, wherein the cells are determined as having immunomodulatory activity using the method of any one of claims 1-5.

10. The method of claim 9, wherein the plurality of cells are determined as having the inflammatory stimulation index (ISI) higher than 1.

11. The method of claim 9 or 10, further comprising administering to the subject a therapeutically effective amount of an anti-COVID therapeutic agent.

12. A method of treating an inflammatory condition in a subject in need thereof, comprising administering to the subject a plurality of cells, wherein the cells are determined as having immunomodulatory activity using the method of any one of claims 1-5.

13. The method of claim 12, wherein the plurality of cells are determined as having the inflammatory stimulation index (ISI) higher than 1.

14. The method of claim 12 or 13, further comprising administering to the subject a therapeutically effective amount of an anti-COVID therapeutic agent.

15. A method of treating a fibrotic condition characterized in a subject in need thereof, comprising administering to the subject a plurality of cells, wherein the cells are determined as having immunomodulatory activity using the method of any one of claims 1-5.

16. The method of claim 15, wherein the plurality of cells are determined as having the inflammatory stimulation index (ISI) higher than 1.

17. The method of claim 15 or 16, further comprising administering to the subject a therapeutically effective amount of an anti-COVID therapeutic agent.

18. A method of treating a condition characterized by an increase in Tumor Necrosis Factor (TNF) in a subject in need thereof, comprising administering to the subject a plurality of cells, wherein the cells are determined as having immunomodulatory activity using the method of any one of claims 1-5.

19. The method of claim 18, wherein the plurality of cells are determined as having the inflammatory stimulation index (ISI) higher than 1.

20. The method of claim 18 or 19, further comprising administering to the subject a therapeutically effective amount of an anti-COVID therapeutic agent.
